(19) United States
(12) Patent Application Publication
(10) Pub. No.: US 2026/0207916 A1
(43) Pub. Date: Jul. 23, 2026

(54) CONNECTION MECHANISM FOR RELEASABLE FLUID-TIGHT CONNECTION OF TWO MEDICAL APPLICANCES

(71) Applicant: Qatna Medical GmbH, Hechingen (DE)

(72) Inventor: Felix Schneider, Albstadt (DE)

(21) Appl. No.: 19/139,014

(22) PCT Filed: Nov. 10, 2023
(86) PCT No.: PCT/EP2023/081384

§ 371 Date: Jun. 13, 2025

(30) Foreign Application Priority Data
Dec. 16, 2022 (DE) 10 2022 133 756.1

(51) Int. Cl. A61M 39/06; A61M 39/02

(52) U.S. Cl. CPC A61M 39/0693; A61M 2039/0258; A61M 2039/0282

(57) ABSTRACT

A connection mechanism for providing a releasable, fluid-tight connection between a first line portion of a first medical appliance and a second line portion of a second medical appliance is disclosed. The connection mechanism includes an engagement element fixed to the first line portion and configured to engage a counter-engagement element on the second line portion in an engagement position. A locking element is movably arranged on the first line portion and is configured to move between a release position and a locking position. In the locking position, the locking element acts on the engagement element to secure it to the counter-engagement element. In the release position, the locking element disengages the engagement element, allowing separation from the counter-engagement element.

The invention relates to a connection mechanism for the releasable fluid-tight connection of two medical appliances.

The percutaneous introduction of a medical appliance into a patient's body vessel is associated with risks, and a variety of methods are known for introducing the medical appliance into the vessel or the body. Common methods include the Seldinger technique.

In each of these methods, the flow of body fluids must be controlled when the medical appliance is inserted into the body vessel. Special care must be taken when introducing it into the blood system. If the vascular pressure is positive, blood can flow out of the medical appliance. The blood flowing out pollutes the surrounding area and can also pose a risk of infection for the doctor. If the vascular pressure is negative, the patient is at risk of air embolism.

Accordingly, medical valves such as hemostatic valves, iris valves, laparoscopic ports, or the like are used to limit or prevent blood loss and air ingress during the procedure.

In addition, medical appliances must also be rinsed with NaCl solution when used in the body, for example, or medications are introduced into the body in liquid form through them; fluid tightness must also be ensured in this application.

Introducer sheaths are medical appliances in themselves and are used in conjunction with other medical appliances such as dilators and cartridges to introduce surgical instruments, implants or active substances into the body and/or into the vascular system of a patient (typically an artery). Introducer sheaths are designed to penetrate the skin and the wall of the blood vessel and can be partially positioned within the blood vessel so that surgical instruments can be introduced and advanced through the introducer sheath from outside the body. In this case, sealing against the surrounding area is particularly problematic.

When two or more medical appliances are connected, there is also a risk of the appliances slipping relative to each other. Slipping can happen in particular unintentionally or due to insufficient securing of the appliances to each other. If it slips, the vessels or surrounding tissue can be injured. In addition, the position of the appliances can change, making it impossible to insert implants or medications. Furthermore, slipping can deteriorate or open a seal between the medical appliances, which can lead to unwanted leakage of body fluids.

EP 2569044 B1 describes an introducer sheath with a hemostatic valve that can maintain hemostasis around surgical instruments with a variety of cross-sectional diameters during surgical procedures.

U.S. Pat. No. 6,551,283 B1 discloses medical appliances and instruments with hemostasis valves and hemostasis cannula units. The hemostasis valve has two separate valve elements, a valve seal and a complementarily shaped valve membrane.

U.S. Pat. No. 9,616,213 B2 relates to medical appliances with adjustable hemostatic valves and systems.

U.S. Pat. No. 9,884,175 B2 shows a medical valve assembly comprising a tube extending between a proximal and a distal tube end.

U.S. 2019/0070394 A1 shows an expandable introduction device and methods for using the same.

U.S. 2018/0256876 A1 shows a medical valve with a variable diameter seal.

The object of the present invention is to provide a connection mechanism for the releasable fluid-tight connection of two medical appliances with increased patient safety.

The object is achieved by a connection mechanism for the releasable, fluid-tight connection of a first line portion of a first medical appliance to a second line portion of a second medical appliance, according to claim 1. In this case, it is provided that the first line portion runs along a first line axis and the second line portion runs along a second line axis. Furthermore, an engagement element is provided which is arranged so as to be stationary on the first line portion and is designed to engage in an engagement position with a counter-engagement element provided on the second line portion. Furthermore, a locking element is provided on the first line portion which can be displaced between a release position and a locking position. In this case, the locking element can be displaced in such a way that it acts on the engagement element in the locking position, so that the engagement element is locked to the counter-engagement element in the engagement position, and that it releases the engagement element in the release position, so that the engagement element can be released from the engagement position.

This configuration enables a secure and defined connectable and releasable connection between two medical appliances. This prevents accidental slipping of a medical appliance into a second medical appliance and increases patient safety. By connecting the line portions, a fluid-tight connection is achieved from which no liquid such as blood, other body fluids or active substances can escape. Even with increased internal pressure in the line portions, the connection remains secure and tight, because the engagement of the engagement element and the action of the locking element prevent the two line portions from moving away from each other. In addition, the connection mechanism increases patient safety when provided on an introducer sheath and a dilator, in particular for vascular interventions when advancing the introducer sheath with the dilator.

Using a connection mechanism according to the invention, even line portions with larger diameters can be connected than in the prior art. Internal diameters of the line portions of more than eleven French can be connected securely and fluid-tight. Preferably, the engagement element has at least one resiliently flexible deflection portion extending obliquely to the first line axis and one engagement portion extending substantially perpendicularly to the first line axis. The deflection portion can be conical at least in portions. The engagement portion is arranged on the free portion of the deflection portion, thereby forming a radial surface. The engagement portion also has a partially cylindrical surface, which can in particular run parallel to the first line axis.

Preferably, the deflection portion and the engagement portion are formed in one piece. Furthermore, the engagement element can be formed in one piece with the line portion or connected to the line portion in another way, such as by adhesive bonding. Furthermore, it is conceivable that the deflection portion and/or the engagement portion are divided into segments which surround the line portion. The resilient flexibility of the deflection portion makes it possible, in particular, for the connection between the first and second medical appliances to be releasable and connectable multiple times.

It is conceivable that the locking element in the locking position acts on the deflection portion in such a way that its resilient flexibility is limited. In particular, the resilient flexibility can be limited outwards in the radial direction so that the engaged state of the engagement element is locked.

This provides a reliable way of securing the connection mechanism.

Preferably, the locking element has an inner cone portion which is at least partially conical and which acts on the deflection portion in the locking position. The inner cone acts on the outside of the deflection portion in such a way that the flexibility is limited in the radial direction outwards. In this case, the outside of the region of the inner cone of the locking element can also be designed to be conical in corresponding portions in order to create a thin-walled structure. A full cone and/or surfaces running obliquely to the axis as the inside of the locking element are also conceivable.

Advantageously, the locking element has an actuating portion which is at least partially cylindrical. The actuating portion is provided for manually moving the locking element. It can also have finger recesses for improved handling.

Particularly preferably, a spring element is provided which urges the locking element towards the engagement element. The locking element is therefore preferably urged into the locking position, whereby it remains there. In order to move it into the release position, it must be moved against a force applied by the spring element. It is also conceivable that the locking element is pushed away from the engagement element so that it remains in the release position. In particular, the spring element can be designed as a helical spring or as an elastomer. Furthermore, it is conceivable that the spring element is formed in one piece with the locking element. Preferably, the spring element is arranged between the actuating portion and the line portion. By applying a spring load to the locking element towards the engagement element, reliable securing and defined positioning of the fluid-tight connection is achieved.

Preferably, the first line portion is designed such that in the engagement position it projects at least partially into the second line portion. This ensures a fluid-tight connection between two line portions. In particular, the end of the second line portion can be closed by a valve element which is penetrated by the first line portion, thus achieving a fluid-tight connection to the surrounding area.

Particularly preferably, the connection mechanism comprises a counter-engagement element arranged so as to be stationary on the second line portion, which counter-engagement element is designed to engage with the engagement element provided on the first line portion, in the engagement position. Preferably, the counter-engagement element can be designed to correspond to the engagement element. The ability to engage the engagement element into the counter-engagement element ensures a secure and fluid-tight connection.

The counter-engagement element preferably has a double cone with a first cone portion which is at least partially conical and a second cone portion which is at least partially conical.

The first cone portion widens along the second line axis to a maximum diameter, and the second cone portion follows the maximum diameter and decreases along the second line axis to a smaller diameter. The first cone portion and the second cone portion are preferably designed as a truncated cone. It is conceivable that the smallest diameter of the second cone portion is larger than the smallest diameter of the first cone portion. Since the counter-engagement element is designed as a double cone, the engagement element can be resiliently deformed in the region of the deflection portion when the connection is established and can at least partially return to its original shape when the locking position is established. Furthermore, the double cone design is advantageous because a connection can be made regardless of the rotational position of the engagement element and the counter-engagement element.

It is conceivable that the second cone portion is followed by a cylinder portion that is at least partially cylindrical. The cylinder portion is suitable for the defined engagement of the cylindrical inner surfaces of the engagement portion.

Preferably, the engagement portion interacts with the second cone portion in the locking position or with the cylinder portion. The second cone portion and/or the cylinder portion can be designed in such a way that the engagement element is resiliently deformed in the engagement position and thus a force is applied which maintains the connection.

Preferably, the engagement element rests, in the engagement position, against the counter-engagement element and/or the deflection portions rest at least partially against the first cone portion. By a defined contact of the inner surfaces with the first cone portion in the engagement position, the connection can be further secured so that even the smallest movements between the engagement element and the counter-engagement element can be prevented during use of the medical appliances.

It is conceivable that a housing portion is provided which is arranged on the second line portion or forms the second line portion, the counter-engagement element being arranged on the housing portion. It is further conceivable that hemostatic valve elements are provided in the housing portion, the design being such that in the engagement position the first line portion penetrates at least partially into the second line portion and penetrates the hemostatic valve elements. Furthermore, the valve elements seal the second line portion fluid-tight against the surrounding area when the second line portion is not connected to the first line portion. The valve elements can be designed as silicone discs with star-shaped incisions or other incisions, the incisions not impairing the sealing effect but ensuring a defined penetration of the first line portion. Preferably, at least two valve elements are provided in the housing portion. Further preferably, the valve elements are arranged rotated relative to one another about the second line axis. This further improves the fluid-tight connection.

Further details and advantageous embodiments of the invention can be found in the following description, on the basis of which embodiments of the invention are described and explained in more detail.

In the drawings:

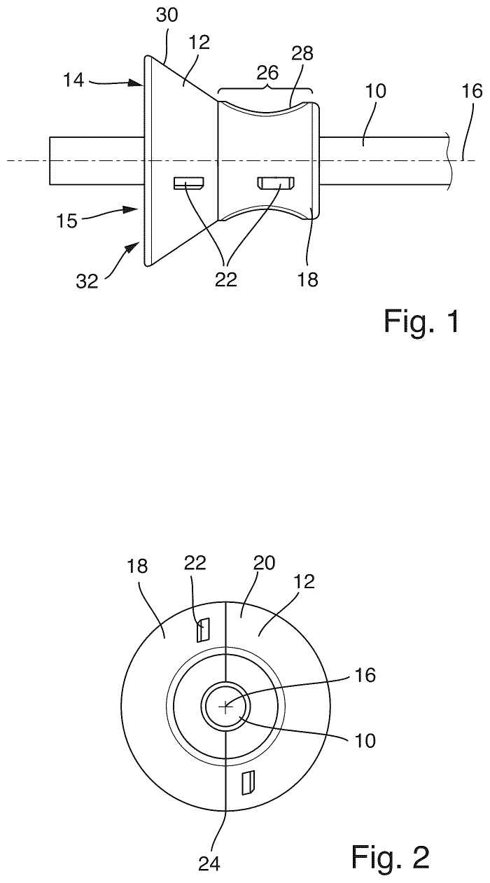

FIG. 1: is a side view of a first line portion with an engagement element and a locking element of a locking mechanism;

FIG. 2: is a rear view of the arrangement of the according to FIG. 1;

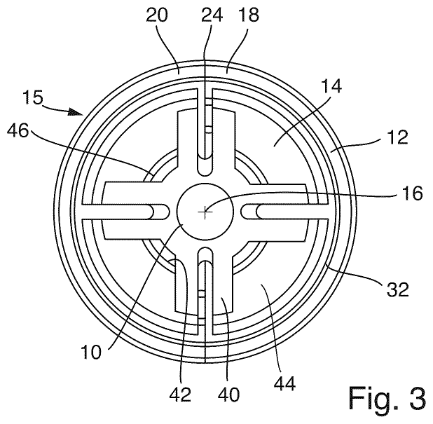

FIG. 3: is a front view of the arrangement according to FIG. 1 into the engagement element;

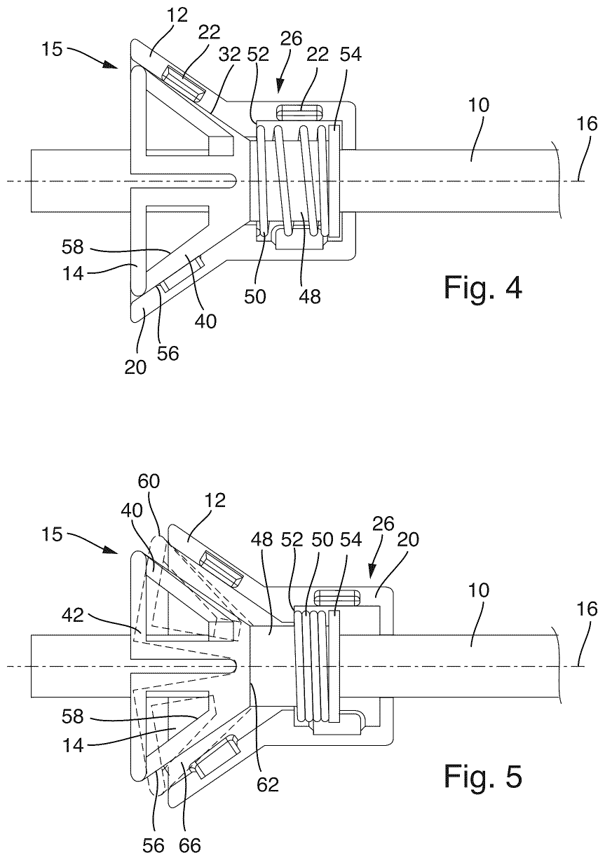

FIG. 4: shows the arrangement according to FIG. 1 with the locking element in the locking position;

FIG. 5: shows the arrangement according to FIG. 3 with the locking element in the release position;

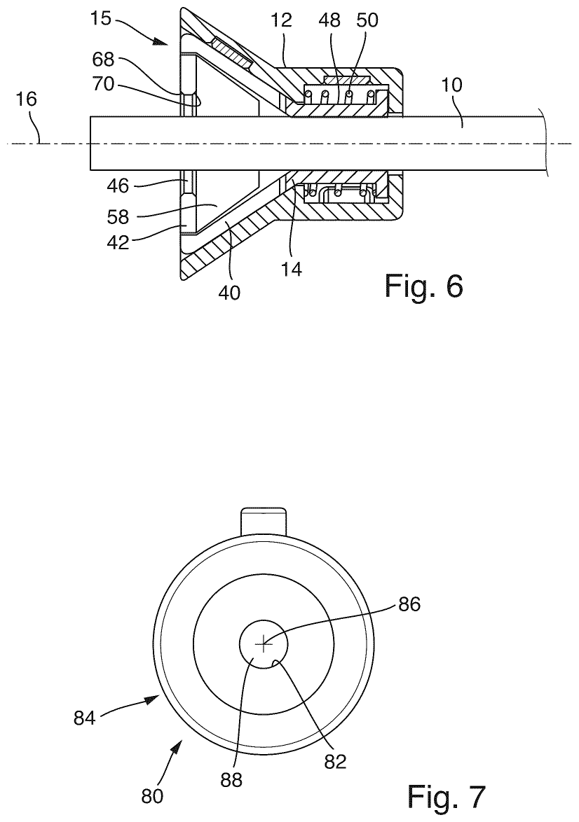

FIG. 6: shows the arrangement according to FIG. 3 with a longitudinally cut engagement element;

FIG. 7: is a front view of a counter-engagement element of the locking mechanism;

FIG. 8: shows the counter-engagement element in a sectional side view with a housing portion and a second line portion;

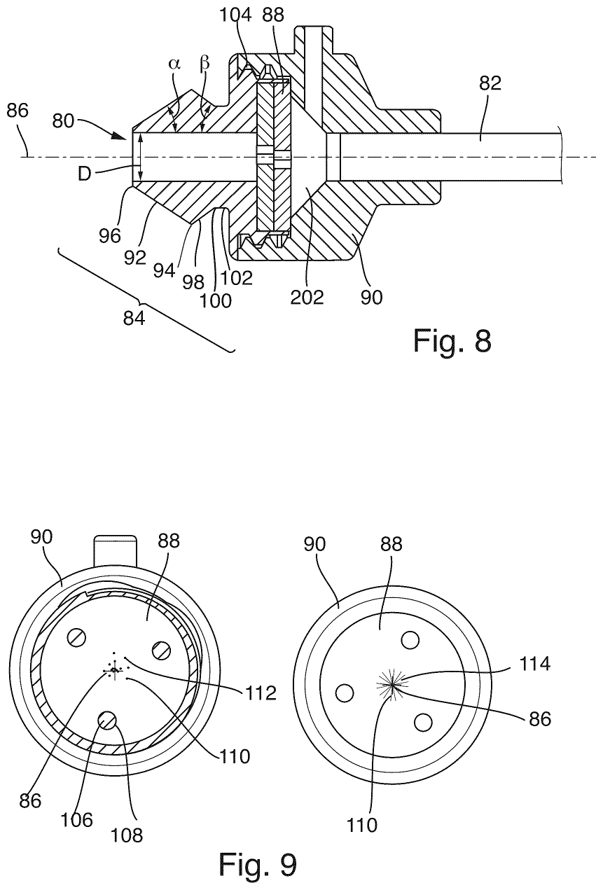

FIG. 9: shows hemostatic valve elements in the housing portion;

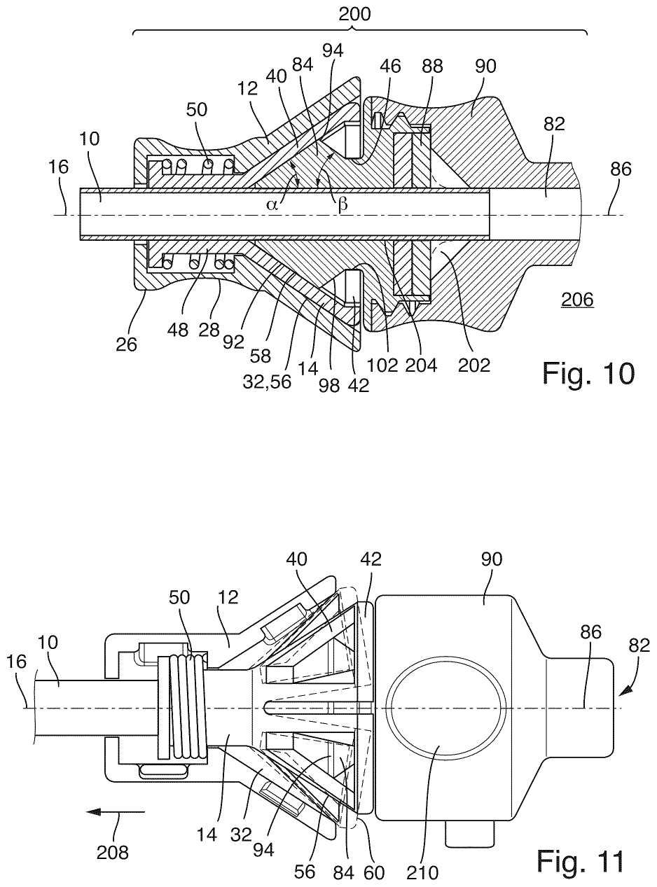

FIG. 10: shows the first line portion connected to the second line portion with the locking element in the locking position;

FIG. 11: shows the arrangement according to FIG. 10 with the locking element in the release position;

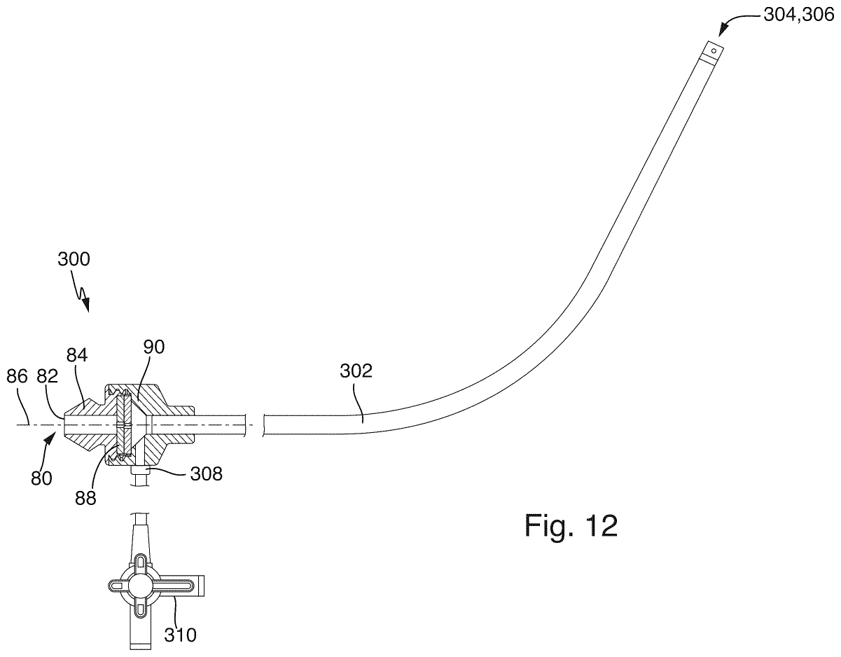

FIG. 12: shows the counter-engagement element arranged on an introducer sheath;

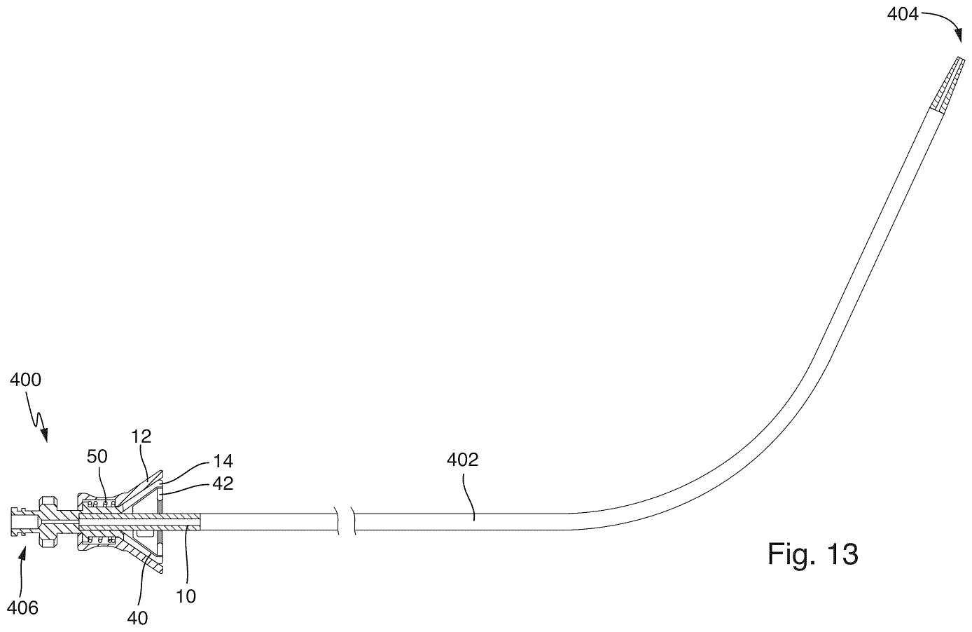

FIG. 13: shows the engagement element arranged on a dilator;

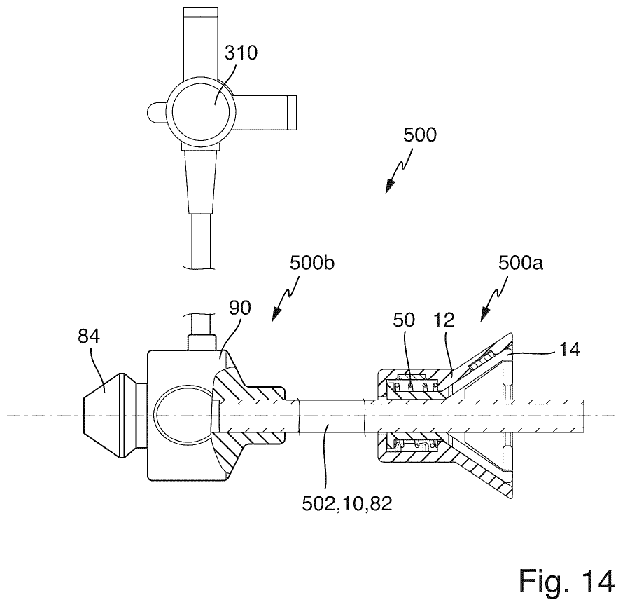

FIG. 14: shows a cartridge with the engagement element and the counter-engagement element arranged at opposite ends of a line portion; and

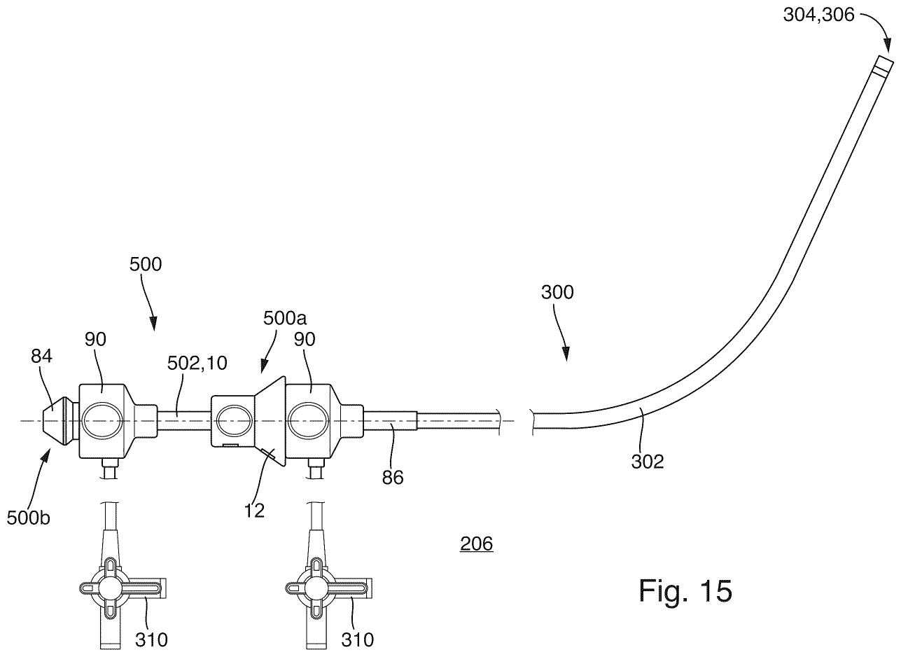

FIG. 15: shows the introducer sheath according to FIG. 12 connected to the cartridge according to FIG. 14 by means of the connection mechanism.

In FIG. 1 and FIG. 2, a first line portion 10 with a locking element 12 is shown in a side view and a rear view. The locking element 12 surrounds an engagement element 14 which is arranged so as to be stationary on the first line portion 10 and which is shown in different views in FIG. 3 to 6. The first line portion 10 is arranged along a first line axis 16 and can be part of a first medical appliance, as shown, for example, in FIGS. 13, 14 and 15 with the reference signs 400 and 500.

The first line portion 10 protrudes from the front side 15 of the arrangement of the locking element 12 and engagement element 14. The locking element 12 is constructed from two connectable parts 18 and 20; however, it is also conceivable to construct the locking element 12 from more than two parts or in one piece (not shown). The parts 18, 20 are designed as half-shells which each surround half of the line portion 10 and are positively connected to one another by means of a clip connection 22. A separation point 24 is provided between the two parts 18, 20, which is located in a plane in which the first line axis 16 lies. The parts 18, 20 can also be joined together by adhesive bonding (not shown).

The locking element 12 is displaceable along the axis 16. For manual displacement, recessed grips 28 are provided in a cylindrically shaped actuating portion 26. With this recessed grip 28, the operator, e.g., a doctor, can safely operate and move the locking element 12. Furthermore, an outer conical portion 30 is provided, which is formed correspondingly to a conical inner portion 32 shown in FIGS. 4, 5 and 6, in order to make the locking element 12 slim and light and to increase the ergonomics.

FIG. 3 shows the arrangement of the first line portion 10 with the engagement element 14 and the locking element 12 in a view from the front along the axis 16 into the locking element 12. The engagement element 14 has a resiliently flexible deformation portion 40 and an engagement portion 42, which is also shown in FIGS. 4, 5 and 6. In this embodiment, the engagement portion 42 and the deformation portion 40 are formed in one piece, although it would also be conceivable to design the deformation portion 40 and the engagement portion in two pieces and/or from different materials. The engagement portion 42 is formed in the radial direction away from the axis 16 and forms a surface 44 towards the front side 15. Furthermore, the engagement portion 42 has an inner contour 46, which can in particular be lip-like. The engagement portion 42 and the deformation portion 40 are divided into four segments which are arranged evenly around the axis 16. However, it would be conceivable to provide only one segment, two or more than four segments and/or not to arrange the segments evenly around the axis 16.

A view according to FIG. 1, but without the half-shell 18 of the locking element 12, is shown in FIG. 4. The engagement element 14 is arranged on the first line portion 10 and is shown surrounded by the locking element 12. A cylindrical portion 48 is arranged adjacent to the deformation portion 40. At this portion 48, the engagement element 14 is arranged so as to be stationary on the first line portion 10; this can be done in particular by adhesive bonding. A helical spring 50 is shown surrounding the portion 48. The helical spring 50 rests on the one hand against the front side 15 on a contact portion 52 on the locking element 12 and on the other hand, facing away from the front side 15, on a circumferential collar 54 on the cylindrical portion 48 of the engagement element 14. In this case, the helical spring 50 is such that it urges the locking element 12 against the engagement element 14 into a locking position. In the locking position, the inner portion 32 rests at least partially against an outer cone portion 56 of the deflection portion 40 and limits or prevents the resilient deformability of the deflection portion 40 radially outward. Furthermore, the deflection portion 40 has an inner cone portion 58.

FIG. 5 shows the view according to FIG. 4 but with the locking element 12 in the release position. The helical spring 50 is compressed and the locking element 12 is retracted along the axis 16 away from the front side 15. Instead of the helical spring 50, other spring elements are conceivable. For example, a spring element can also be formed in one piece with the locking element 12 or the engagement element 14 (not shown).

In the release position, the inner portion 32 does not rest against the outer cone portion 56, so that the deflection portion 40 can be resiliently deflected radially outwards according to the dashed lines 60. For this purpose, the entire deflection portion 40 can be deformed or the deflection portion 40 forms a solid joint in the region of the transition 62 into the portion 48. Window-like material recesses 64 are provided in the region of the deflection portion 40, so that only thin webs 66 remain and these contribute to the deformability. Furthermore, instead of the conical regions 32, 56, 58 and partially rotationally symmetrical geometries of the deflection portion 40 and the locking element 12, it is also conceivable to provide simple corresponding surfaces running obliquely to the axis 16 (not shown).

FIG. 6 shows a view according to FIG. 4, the engagement element 14 being shown in section. The inner contour 42 on the engagement portion 42 is clearly shown. This is preferably rounded at the edges 68, 70 facing away from and towards the front side 15 in order to improve sliding over surfaces of a corresponding counter-engagement element 84 (shown in FIGS. 7 and 8).

A counter-engagement element 84 is shown in various views and sections in FIG. 7 to 9. A view of a front side 80 into the counter-engagement element 84 surrounding a second line portion 82 is shown in FIG. 7. The second line portion 82 is formed along a second line axis 86, it being possible for the second line portion 82 to be part of a second medical appliance or to be partially formed by the medical appliance and/or by the counter-engagement element 84.

The second medical appliance is shown in FIG. 12 in the form of an introducer sheath 300 and in FIG. 14 in the form of a cartridge 500. Furthermore, the introducer sheath 300 and the cartridge are shown connected to each other in FIG. 15. Hemostatic valve elements 88 are arranged in the line portion 82 for sealing the second line portion 82.

FIG. 8 shows a longitudinal section through the counter-engagement element 84 and a housing portion 90 on which the counter-engagement element 84 is arranged. The counter-engagement element 84 has a first cone portion 92 which widens from the front side 80 along the axis 86 from the diameter D of the line portion 82 to a maximum diameter 94. However, the minimum diameter 96 of the first cone portion 92 can also be larger than the diameter D. A second cone portion 98 is arranged directly at the maximum diameter 94. The second cone portion 98 tapers along the axis 86 to a minimum diameter 100. This is followed by a cylindrical portion 102 with a diameter 100. Instead of the first cone portion 92, the second cone portion 98 and the cylindrical portion 102, other non-rotationally symmetrical geometries are also conceivable, for example surfaces running obliquely to the axis 86 (not shown).

The counter-engagement element 84 can be arranged on the housing portion 90 by means of a connection element 104. The connection element 104 can in particular be a thread; other types of connection such as clips or clamps are conceivable. In addition, the valve elements 88 can be clamped fluid-tight by assembling, in particular screwing or clamping, the counter-engagement element 84 to the housing portion 90. At least one valve element 88, but preferably two valve elements 88, are provided to ensure a secure seal.

FIG. 9 shows various hemostatic valve elements 88 arranged in the housing portion 90, with the counter-engagement element 84 unscrewed and not shown. For the torsion-proof arrangement of the valve elements 88, pin-like elements 106 are provided in the housing portion 90, which correspond to corresponding holes 108 in the valve elements 88. Self-sealing passages 110 are provided in the valve elements 88 in the region of the second line portion 82 around the axis 86, which passages enable the first line portion 10 or other medical appliance 400, 500a to pass through in a fluid-tight manner. The incisions may be formed as perforations 112 or star-shaped incisions 114. Furthermore, the valve elements 88 can be arranged offset according to the axis 86 in order to ensure further improved sealing.

FIG. 10 shows a longitudinal section with the first line portion 10, which is connected in a fluid-tight manner to the second line portion 82 by the connection mechanism 200. The engagement element 14 is in the engagement position with the counter-engagement element 84. The inner cone portion 58 rests against the first cone portion 92. The engagement portion 42 rests with the inner contour 46 on the cylindrical portion 102. The deformation portion 40 can be deformed in the engagement position, since the diameter 100 can be larger than the diameter of the inner contour 46. The locking element 12 is urged by the spring 50 against the outer cone portion 56 of the deformation portion 40, so that further deformation of the deformation portion 40 is not possible. The connection is therefore not releasable and does not allow any relative movement along the axes 16, 86 between the line portions 10, 82.

Due to the at least partially rotationally symmetrical structure of the engagement element 14, the counter-engagement element 84 and the locking element 12, the components 12, 14, 84 can be rotated relative to one another about the axes 16, 86, even when the connection mechanism 200 is in the engagement position and the connection is established. Even if twisted, the connection remains fluid-tight. This is advantageous when the position needs to be changed while inserted into the body. Furthermore, the connection 200 can be made and released independently of the rotational position about the axes 16, 86. The first line axis 16 is congruent with the second line axis 86 in the region of the connection mechanism 200. The first line portion 10 projects into the second line portion 82 and penetrates the valve elements 88, which can be penetrated and/or displaced into a cavity 202. This improves the sealing and simplifies the insertion of the first line portion 10. The valve elements 88 are at least partially in contact with the outside 204 of the first line portion 10 with the passages 110 and seal the connection to the surrounding area 206. A second cavity (not shown) could be provided on the opposite side of the cavity 202, facing the front side 80. This second cavity would simplify the withdrawal of the first line portion 10 when separating the connection and reduces wear of the valve elements 88.

FIG. 11 shows the view according to FIG. 10, but with the locking element 12 displaced into the release position. The spring 50 is compressed. To release the connection, the locking element must be moved in the direction of arrow 208 into the release position from the locking position as shown in FIG. 10. The recessed grips 28 and/or the actuating portion 26 are suitable for pulling the locking element. This releases the engagement element 12. If the pulling continues in direction 208, the deflection portion 40 is resiliently deflected radially outwards according to the lines 60 due to the surface contact of the inner contour 46 and the second cone portion 98 and the connection is released from the engagement position. As soon as the engagement portion 42 with the inner contour 46 has overcome the maximum diameter 94, the deflection portion 40 again assumes its initial position according to FIG. 4. If the pulling continues in direction 208, the first line portion 10 slides out of the second line portion 82 and the valve elements 88 and the connection is separated. In the separated state, the second line portion 88 is sealed against the surrounding area 206 by the valve elements 88.

For further improved handling, recessed grips 210 are also provided on the housing portion 90. This is advantageous when the housing portion 90 is arranged on or forms a medical appliance and it is inserted into the body and is not to be pulled out when the connection is separated. To establish the connection, the first line portion 10 must first be inserted into the second line portion 86 in the counter-engagement element 84 in the opposite direction to the direction 208, then the locking element 12 must be retracted into the release position in the direction of arrow 208, so that the deformability of the deflection portion 40 is enabled. The deflection portion 40 is deformed by the first cone portion 92 and at least partially reshaped into the engagement position by the second cone portion 98. As soon as the engagement element 14 is in the engagement position, the locking element 12 can be released so that the spring 50 pushes it against the engagement element 14 into the locking position and locks it as described. To further improve the handling of the connection mechanism 200, it is conceivable that the arrangement of locking element 12, engagement element 14 and spring 50 is coordinated in such a way that the locking element 12 does not have to be retracted to establish the connection. In such an embodiment, pushing the engagement element 14 onto the first cone portion 92 causes both a deformation of the engagement element 14 and a displacement of the locking element 12, so that a prior retraction of the locking element 12 is not necessary. However, to separate the connection, the locking element 12 must be retracted. This can be achieved by different angles α of the first cone portion 92 and angles β of the second cone portion 98 with respect to the axis 86.

FIG. 12 shows a second medical appliance in the form of an introducer sheath 300 with the housing portion 90, with the valve elements 88 and the counter-engagement element 84. The second line portion 82 is partially formed by the counter-engagement element 84 and the housing portion 90. An insertion tube 302 is arranged on the housing portion 90 facing away from the front side 80 of the counter-engagement element 84, which tube continues the line portion 86 to its end 304. The line portion 86 opens into an opening 306 at the free end 304. The insertion tube 302 can have different lengths and inner diameters depending on the purpose. Furthermore, it can be pre-formed for the desired purpose and made rigid or flexible. In the housing portion 90, a branch 308 from the line portion 86 is provided, on which a three-way valve 310 is arranged. By means of the three-way valve 310, the line portion 86 can be rinsed, active substances can be introduced, and liquids can be sucked off and aspirated.

FIG. 13 shows a first medical appliance in the form of a dilator 400 having a dilator body 402 and a tip 404 for dilating a channel in tissue upon insertion into a body. The engagement element 14 and the locking element 12 are arranged on the dilator body 402, the dilator body 402 forming the first line portion 10. Furthermore, a Luer connector 406 is arranged on the engagement element 14, which continues the line portion 10. The line portion 10 is continuous from the Luer connector 406 to the tip 404. A syringe (not shown) for injection, in particular for rinsing, can be connected to the Luer connector 406. Furthermore, a guide wire (not shown) can be inserted into the line portion 10 of the dilator 300 for insertion into a body.

The dilator 400 is suitable for being connected to the introducer sheath 300 by means of the connection mechanism 200 in order to safely introduce the introducer sheath 300 into a body. The dilator 400 also prevents the insertion tube 302 from kinking during insertion into the body. After insertion, the dilator 300 can be safely separated from the introducer sheath 300 by means of the connection mechanism 200 and withdrawn without leakage of body fluids and/or the risk of air embolism.

FIG. 14 shows a cartridge 500 comprising a first medical appliance 500a and a second medical appliance 500b. One end forms the first medical appliance 500a and the other end forms the second medical appliance 500b, wherein the engagement element 14, the locking element 12, the counter-engagement element 84 and the housing portion 90 are arranged on a common line portion 502. For the line portion 502, what was said for the line portion 10 applies in the region of the engagement element 14, and what was said for the line portion 82 applies in the region of the housing portion 90 and counter-engagement element 84. The cartridge 500 is suitable for being connected to the introducer sheath 300 by means of the connection mechanism 200. The cartridge 500 can receive active substances or implants in the line portion 502, which can be introduced into the body at a specific anatomical position by means of the introducer sheath 300. Such a cartridge 500 with components of the connection mechanism 200 makes it possible to connect further medical appliances such as a further cartridge 500 and thus to extend the introducer sheath 300 in a fluid-tight manner against the surrounding area 206.

FIG. 15 shows the cartridge 500 connected to the introducer sheath 300 by means of the connection mechanism 200. The locking element 14 is in the locking position. The common line portion 502 can be considered the first line portion 10 and is connected in fluid-tight manner and securely to the second line portion 86. To release the connection, the same applies as described in relation to FIGS. 10 and 11. It can be seen that a further first medical appliance 500a, 400, comprising an arrangement of engagement element 14, locking element 12 and line portion 10, can be arranged on the free engagement element 84 at the proximal end of the cartridge 500 on the second medical appliance 500b. It is also conceivable that two or more cartridges 500 can be connected to each other and arranged one behind the other.

With the connection mechanism 200 shown, it is possible to connect various medical appliances, such as an introducer sheath 300, a dilator 400 and/or a cartridge 500, in a fluid-tight and secure manner and to release the connection again. Furthermore, the connection can be established, released and/or the medical appliances can be rotated relative to one another regardless of the rotational position of the medical appliances with respect to their axes 10, 86, 502. This increases the safety of the patient using medical appliances 300, 400, 500 with the connection mechanism 200.

1.-13. (canceled)

14. A connection mechanism for releasable fluid-tight connection of a first line portion, extending along a first line axis, of a first medical appliance to a second line portion, extending along a second line axis, of a second medical appliance, the connection mechanism comprising:
an engagement element arranged to be stationary on the first line portion and configured to engage with a counter-engagement element provided on the second line portion in an engagement position; and
a locking element displaceable on the first line portion and movable between a release position and a locking position,
wherein, in the locking position, the locking element acts on the engagement element to lock the engagement element on the counter-engagement element, and
wherein, in the release position, the locking element releases the engagement element so that the engagement element can be released from the engagement position,
wherein the engagement element includes at least one resiliently flexible deflection portion that is conical at least in part and extends obliquely to the first line axis, and an engagement portion extending substantially perpendicularly to the first line axis,
wherein the locking element includes an inner cone portion that is at least partially conical and configured to act on the deflection portion in the locking position,
wherein the counter-engagement element includes a double cone having a first cone portion that is at least partially conical and a second cone portion that is at least partially conical,
wherein the first cone portion widens along the second line axis to a maximum diameter, and the second cone portion follows the maximum diameter and decreases along the second line axis to a smaller diameter, and
wherein, in the engagement position, the engagement element rests against the counter-engagement element and the deflection portion rests at least partially against the first cone portion.

15. The connection mechanism of claim 14, wherein the locking element in the locking position acts on the deflection portion in such a way that the resilient flexibility of the deflection portion is limited.

16. The connection mechanism of claim 14, wherein the locking element includes an actuating portion that is at least partially cylindrical.

17. The connection mechanism of claim 14, further comprising a spring element that urges the locking element toward the engagement element.

18. The connection mechanism of claim 17, wherein the first line portion is configured such that, in the engagement position, the first line portion projects at least partially into the second line portion.

19. The connection mechanism of claim 14, wherein the counter-engagement element is arranged to be stationary on the second line portion and is configured to engage with the engagement element in the engagement position.

20. The connection mechanism of claim 14, wherein the second cone portion is adjoined by an at least partially cylindrical cylinder portion.

21. The connection mechanism of claim 14, wherein, in the locking position, the engagement portion interacts with the second cone portion.

22. The connection mechanism of claim 20, wherein, in the locking position, the engagement portion interacts with the cylinder portion.

23. The connection mechanism of claim 14, further comprising a housing portion arranged on the second line portion or forming the second line portion,
wherein the counter-engagement element is arranged on the housing portion,
wherein hemostatic valve elements are provided in the housing portion, and
wherein, in the engagement position, the first line portion penetrates at least partially into the second line portion and penetrates the hemostatic valve elements.
